(19) United States
(12) Patent Application Publication
(10) Pub. No.: US 2026/0345998 A1
(43) Pub. Date: Nov. 5, 2026

(54) DEFIBRILLATOR ELECTRODE PAD

(71) Applicant: Nihon Kohden Corporation, Tokyo (JP)

(72) Inventors: Yuya Komeiji, Saitama (JP); Ryugo Odaka, Saitama (JP); Takeshi Akiyama, Saitama (JP)

(21) Appl. No.: 19/124,735

(22) PCT Filed: Oct. 13, 2023
(86) PCT No.: PCT/JP2023/037211

§ 371 Date: Apr. 25, 2025

(30) Foreign Application Priority Data
Oct. 28, 2022 (JP) 2022-173469

(51) Int. Cl. A61N 1/04

(52) U.S. Cl. CPC A61N 1/046; A61N 1/048; A61N 1/0496

(57) ABSTRACT

A defibrillator electrode pad includes a metal foil configured to diffuse electricity transmitted from a defibrillator via a cable, a conductive adhesive layer configured to be attached to a living body, the conductive adhesive layer being provided on a part of a first surface of the metal foil, a base member provided on a second surface of the metal foil, and an insulating part covering a portion, of the first surface, at which the conductive adhesive layer is not provided, the insulating part fixing, to the metal foil, a cable terminal provided at an end portion of the cable.

### CROSS-REFERENCE TO RELATED APPLICATIONS

This application is a 371 national phase entry of International Patent Application No. PCT/JP2023/037211, filed Oct. 13, 2023, entitled “DEFIBRILLATOR ELECTRODE PAD,” which in turn claims priority to Japanese Patent Application No. 2022-173469, filed Oct. 28, 2022, each of which is incorporated by reference herein, in the entirety and for all purposes.

### TECHNICAL FIELD

The presently disclosed subject matter relates to a defibrillator electrode pad.

### BACKGROUND

The related art discloses a technique related to an electrode pad attached to a living body when a defibrillator such as an automated external defibrillator (AED) is used. For example, an electrode for a living body described in PLT 1 includes a conductive gel, an electrode element made of metal and provided on one surface of the conductive gel, and a cable for electrically connecting the electrode for a living body and an external device, and the electrode element and a terminal attached to an end of the cable is connected using a rivet or grommet made of metal.

### CITATION LIST

JP2013-192818A

### SUMMARY

### Technical Problem

In the electrode for a living body disclosed in PLT 1, as described above, the terminal of the cable and the electrode element are fastened to each other by a rivet or grommet made of metal, and the rivet is covered with a label in order to avoid exposure of the rivet. Meanwhile, there is a demand for a defibrillator electrode pad in which the number of components is reduced and exposure of a metal component is avoided.

An object of the presently disclosed subject matter is to provide a defibrillator electrode pad in which the number of components is reduced and exposure of a metal component is avoided.

### Solution to Problem

A defibrillator electrode pad includes:
a metal foil configured to diffuse electricity transmitted from a defibrillator via a cable;
a conductive adhesive layer configured to be attached to a living body, the conductive adhesive layer being provided on a part of a first surface of the metal foil;
a base member provided on a second surface of the metal foil; and
an insulating part covering a portion, of the first surface, at which the conductive adhesive layer is not provided, the insulating part fixing, to the metal foil, a cable terminal provided at an end portion of the cable.

### Effects of the Invention

According to the presently disclosed subject matter, the number of components is reduced and exposure of the metal component is avoided.

### BRIEF DESCRIPTION OF DRAWINGS

FIG. 1 is an exploded perspective view illustrating an overall configuration of a defibrillator electrode pad according to a first embodiment of the presently disclosed subject matter.

FIG. 2 illustrates a state where the defibrillator electrode pad illustrated in FIG. 1 is assembled.

FIG. 3 illustrates a shape of an outer surface of a first insulating portion illustrated in FIG. 1.

FIG. 4 illustrates a shape of an inner surface of the first insulating portion illustrated in FIG. 1.

FIG. 5 illustrates a shape of an outer surface of a second insulating portion illustrated in FIG. 1.

FIG. 6 illustrates a shape of an inner surface of the second insulating portion illustrated in FIG. 1.

FIG. 7 is a cross-sectional view illustrating a configuration of a defibrillator electrode pad according to a second embodiment of the presently disclosed subject matter.

FIG. 8 illustrates Modification 1 of a base member and a covering portion illustrated in FIG. 7.

FIG. 9 illustrates Modification 2 of the base member and the covering portion illustrated in FIG. 7.

FIG. 10 illustrates a configuration of a defibrillator electrode pad according to a third embodiment of the presently disclosed subject matter.

FIG. 11 illustrates a configuration of a first snap and a second snap illustrated in FIG. 10.

FIG. 12 illustrates a configuration of a first snap and a second snap according to a modification of the third embodiment of the presently disclosed subject matter.

### DESCRIPTION OF EMBODIMENTS

Hereinafter, an example of an embodiment of a defibrillator electrode pad according to the presently disclosed subject matter will be described with reference to the accompanying drawings.

### First Embodiment

### (Overall Configuration)

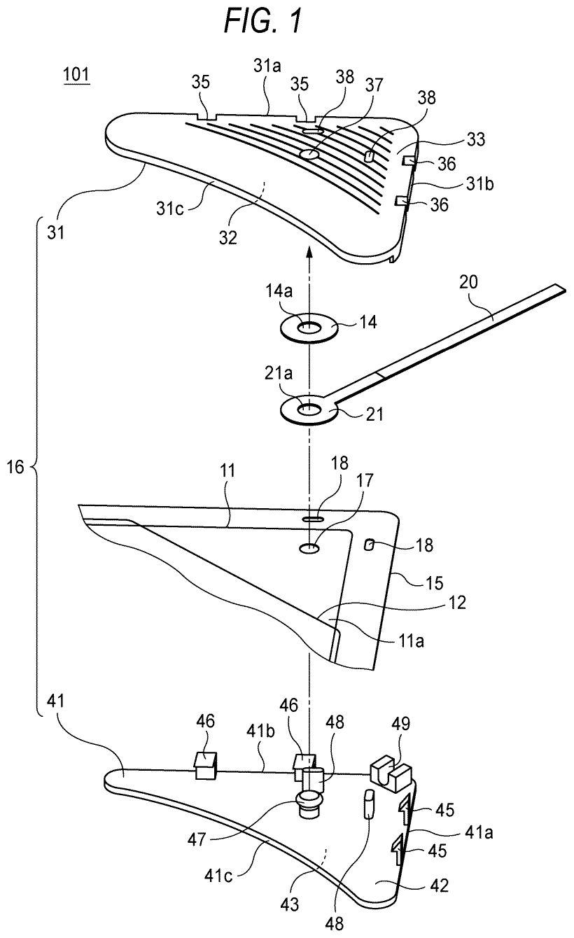

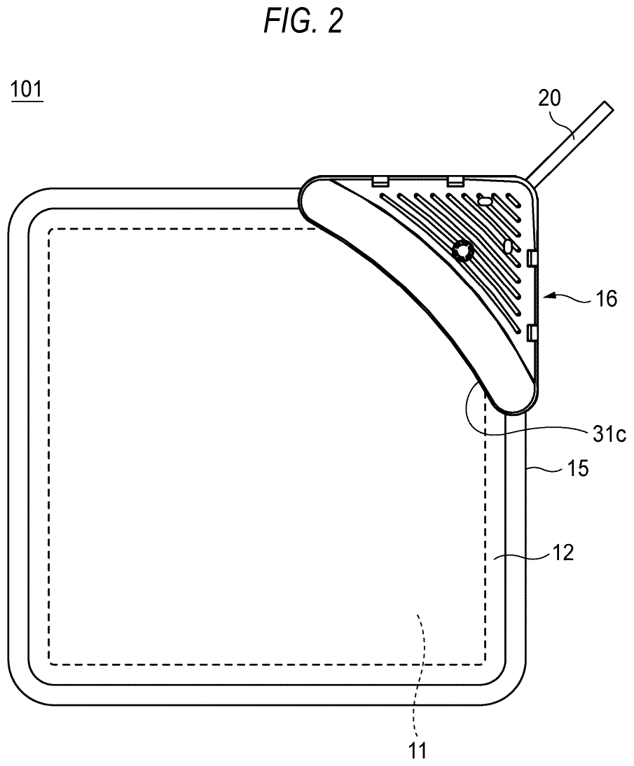

FIG. 1 is an exploded perspective view illustrating an overall configuration of a defibrillator electrode pad 101 according to a first embodiment of the presently disclosed subject matter. FIG. 2 illustrates a state where the defibrillator electrode pad 101 illustrated in FIG. 1 is assembled.

With reference to FIGS. 1 and 2, the defibrillator electrode pad 101 is an electrode pad configured to be attached to a living body in a case where a defibrillator such as an automated external decompressor (AED) is used. The defibrillator electrode pad 101 can include a metal foil 11, a conductive adhesive layer 12, a washer 14, a base member 15, an insulating part 16, and a release sheet, which is not illustrated. A defibrillator and the defibrillator electrode pad 101 are configured to be electrically connected via a cable 20.

The metal foil 11 is, for example, a tin foil. The metal foil 11 is configured to diffuse electricity transmitted from the defibrillator via the cable 20. The metal foil 11 has, for example, a substantially square shape or rectangular shape having corners. The corners may be chamfered or rounded. The metal foil 11 illustrated in FIG. 2 has a substantially square shape, and corners thereof are rounded. The metal foil 11 is not limited to a shape having corners, and may have a shape having no corner, such as an elliptical shape.

As illustrated in FIG. 1, the conductive adhesive layer 12 is attached to a part of one surface (hereinafter referred to as a “first surface 11a”) of the metal foil 11. The base member 15 is attached to the other surface of the metal foil 11, that is, a surface opposite to the first surface 11a. The base member 15 has, for example, a substantially square or rectangular shape larger than the metal foil 11, and corners thereof are rounded.

The conductive adhesive layer 12 is, for example, a gel having viscosity., and the release sheet is attached to the conductive adhesive layer 12 in a case where the defibrillator is not used. In a case where the defibrillator is used, an operator peels off the release sheet from the conductive adhesive layer 12 and attaches the conductive adhesive layer 12 to a living body.

A cable terminal 21 provided on an end portion of the cable 20 has, for example, a circular shape and has a circular hole 21a inside. The washer 14 is made of an elastic resin or the like. The washer 14 has a circular hole 14a inside the same as or similarly to the cable terminal 21.

The insulating part 16 covers a portion of the first surface 11a of the metal foil 11 to which the conductive adhesive layer 12 is not attached. For example, as illustrated in FIG. 1, it is assumed that the conductive adhesive layer 12 is not attached to one of the corners of the metal foil 11. In this case, the insulating part 16 covers the corner of the metal foil 11. Therefore, as illustrated in FIG. 2, the metal foil 11 is not exposed in a state where the defibrillator electrode pad 101 is assembled.

The insulating part 16 fixes the cable terminal 21 illustrated in FIG. 1 to the metal foil 11. Specifically, the insulating part 16 can include a first insulating portion 31 that covers the first surface 11a of the metal foil 11, and a second insulating portion 41 configured to be fitted to the first insulating portion 31. The first insulating portion 31 and the second insulating portion 41 are fitted to each other in a state where the metal foil 11, the cable terminal 21, and the washer 14 are sandwiched between the first insulating portion 31 and the second insulating portion 41. Thus, the cable terminal 21 is fixed to the metal foil 11.

### (Configuration of First Insulating Portion)

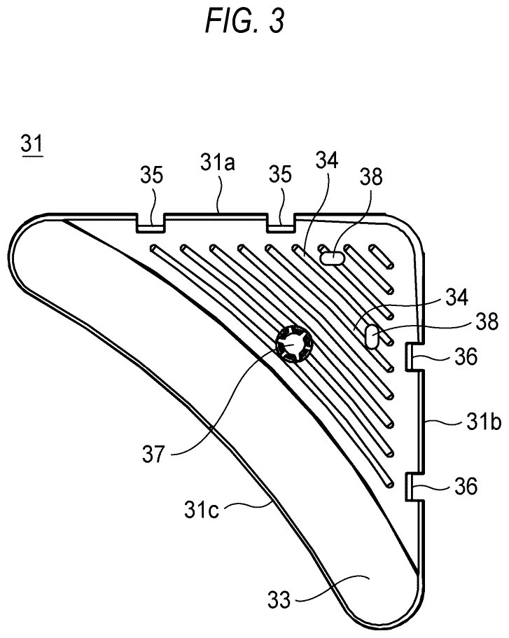

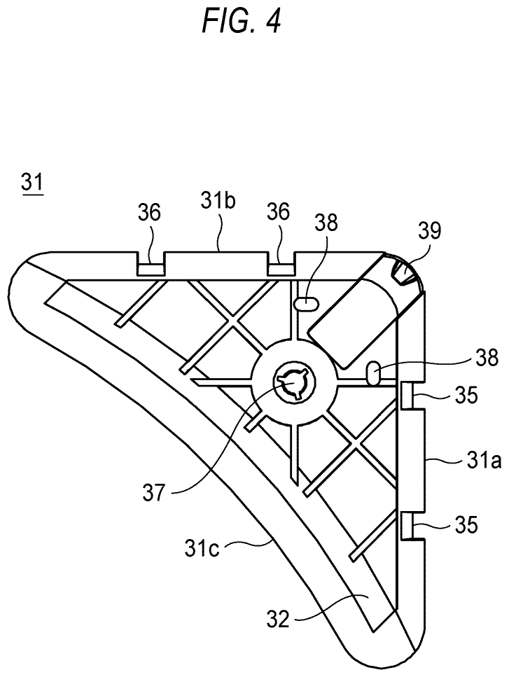

The first insulating portion 31 has, for example, a substantially isosceles right triangular shape. The first insulating portion 31 can include an inner surface 32 in contact with the metal foil 11 and an outer surface 33 opposite to the inner surface 32. FIG. 3 illustrates a shape of the outer surface 33 of the first insulating portion 31 illustrated in FIG. 1. FIG. 4 illustrates a shape of the inner surface 32 of the first insulating portion 31 illustrated in FIG. 1.

Referring to FIGS. 3 and 4, the first insulating portion 31 is preferably made of a material that has strength and elasticity, that does not undergo hydrolysis even in a case where in contact with moisture, and that does not crack even at a high temperature. For example, the first insulating portion 31 is made of a resin such as polyacetal (POM) or polyethylene terephthalate (PET).

The first insulating portion 31 has two sides 31a and 31b sandwiching a right angle corner, and a side 31c facing the right angle corner. As illustrated in FIG. 2, in a state where the first insulating portion 31 covers the metal foil 11, that is, in a state where the defibrillator electrode pad 101 is assembled, the side 31c is positioned on a center side of the metal foil 11. The side 31c is curved, for example, in a direction away from a center of the metal foil 11, that is, toward a center side of the first insulating portion 31.

As illustrated in FIG. 3, an anti-slip processing is applied to the outer surface 33 of the first insulating portion 31. Specifically, a plurality of protrusions 34 extending in a direction along the side 31c are formed on the outer surface 33, as an example of the anti-slip processing.

The first insulating portion 31 can include an engaging portion 35 formed on the side 31a, an engaging portion 36 formed on the side 31b, a hole 37 formed in the vicinity of the center of the first insulating portion 31, and a hole 38 formed closer to a right angle corner side with respect to the center of the first insulating portion 31. The first insulating portion 31 illustrated in FIGS. 3 and 4 can include, for example, two engaging portions 35, two engaging portions 36, one hole 37, and two holes 38. Further, as illustrated in FIG. 4, a groove 39 configured to hold the cable 20 illustrated in FIG. 1 is provided at a right angle corner of the first insulating portion 31.

### (Configuration of Second Insulating Portion)

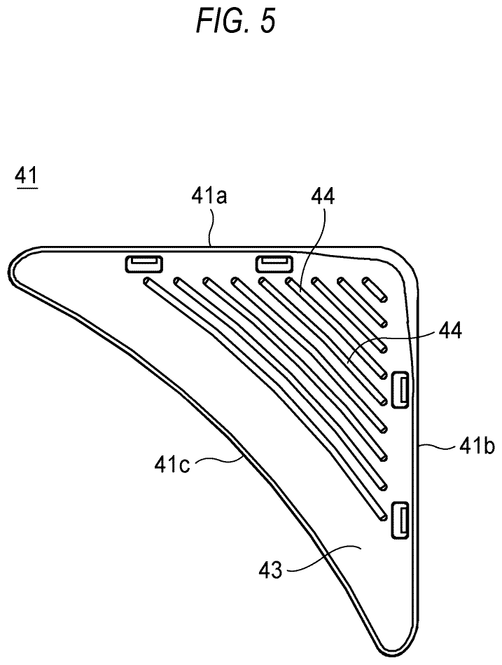

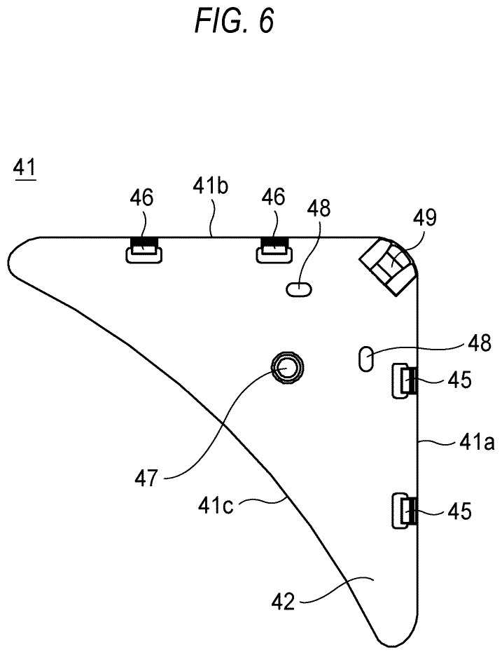

Referring again to FIG. 1, the same as or similarly to the first insulating portion 31, the second insulating portion 41 has, for example, a substantially isosceles right triangular shape. The second insulating portion 41 has an inner surface 42 in contact with the base member 15, and an outer surface 43 opposite to the inner surface 42. FIG. 5 illustrates a shape of the outer surface 43 of the second insulating portion 41 illustrated in FIG. 1. FIG. 6 illustrates a shape of the inner surface 42 of the second insulating portion 41 illustrated in FIG. 1. Referring to FIGS. 5 and 6, the same as or similarly to the first insulating portion 31, the second insulating portion 41 is made of, for example, a resin such as POM or PET.

The same as or similarly to the first insulating portion 31, the second insulating portion 41 has two sides 41a and 41b sandwiching a right angle corner, and a side 41c facing the right angle corner. As illustrated in FIG. 1, the side 41c is positioned on the center side of the metal foil 11 in a state where the defibrillator electrode pad 101 is assembled. The side 41c is curved, for example, in a direction away from the center of the metal foil 11, that is, toward a center side of the second insulating portion 41.

As illustrated in FIG. 5, an anti-slip processing is applied to the outer surface 43 of the second insulating portion 41. Specifically, a plurality of protrusions 44 extending in a direction along the side 41c are formed on the outer surface 43, as an example of the anti-slip processing.

As illustrated in FIG. 6, the second insulating portion 41 can include a claw portion 45 formed near the side 41a, a claw portion 46 formed near the side 41b, a protruding portion 47 formed in the vicinity of a center of the second insulating portion 41, and a protruding portion 48 formed closer to a right angle corner side with respect to the center of the second insulating portion 41. The second insulating portion 41 illustrated in FIG. 6 can include, for example, two claw portions 45, two claw portions 46, one protruding portion 47, and two protruding portions 48. Further, a groove 49 configured to hold the cable 20 illustrated in FIG. 1 is provided at a right angle corner of the second insulating portion 41.

### (Assembly of Defibrillator Electrode Pad)

Referring again to FIG. 1, a hole 17 penetrating the metal foil 11 and the base member 15 is formed in the portion of the metal foil 11 to which the conductive adhesive layer 12 is not attached. Further, two holes 18 penetrating the base member 15 are formed in the base member 15 at a corner near the hole 17.

During assembly of the defibrillator electrode pad 101, the protruding portion 47 of the second insulating portion 41 is inserted into the hole 17 of the base member 15, the hole 21a of the cable terminal 21, and the hole 14a of the washer 14, in a state where the inner surface 42 of the second insulating portion 41 faces upward. Further, at this time, the two protruding portions 48 of the second insulating portion 41 are inserted into the respective two holes 18 of the base member 15. In this state, the two claw portions 45, the two claw portions 46, and the groove 49 of the second insulating portion 41 are located on an outer side of the base member 15. The protruding portions 48 are exposed from the holes 18.

Further, the cable 20 is disposed between the two protruding portions 48, and passes through the inside of the groove 49. Then, the first insulating portion 31 and the second insulating portion 41 are fitted to each other, in a state where the inner surface 32 of the first insulating portion 31 faces downward.

More specifically, the protruding portion 47 of the second insulating portion 41 is inserted into the hole 37 of the first insulating portion 31. Here, for example, a plurality of notches are formed on an outer periphery of the hole 37 of the first insulating portion 31 (see FIG. 3). Further, claws extending radially are formed at a tip end of the protruding portion 47 of the second insulating portion 41. In a case where the claws of the protruding portion 47 are inserted into the hole 37 while widening the notches of the hole 37 and the claws reach the outer surface 33 of the first insulating portion 31, the notches of the hole 37 are narrowed, and the protruding portion 47 is not detached from the hole 37.

The two protruding portions 48 of the second insulating portion 41 are inserted into the respective two holes 38 of the first insulating portion 31. Further, the two claw portions 45 of the second insulating portion 41 are engaged with the respective two engaging portions 36 of the first insulating portion 31, and the two claw portions 46 of the second insulating portion 41 are engaged with the respective two engaging portions 35 of the first insulating portion 31. Accordingly, the first insulating portion 31 and the second insulating portion 41 are fitted to each other, and the assembly of the defibrillator electrode pad 101 is completed.

The first insulating portion 31 and the second insulating portion 41 are not limited to having the isosceles right triangular shape. The first insulating portion 31 and the second insulating portion 41 are not limited to the configuration in which the first insulating portion 31 and the second insulating portion 41 are provided at the corner of the metal foil 11, and may be provided, for example, near a center of a side of the metal foil 11.

Further, the first insulating portion 31 and the second insulating portion 41 may be colored in different colors. Accordingly, the first insulating portion 31 and the second insulating portion 41 can be easily distinguished from each other by an operator who assembles the defibrillator electrode pad 101. The first insulating portion 31 and the second insulating portion 41 may have different sizes, and for example, the first insulating portion 31 may have a smaller shape than the second insulating portion 41.

Further, the first insulating portion 31 and the second insulating portion 41 are not limited to those formed of a resin, and may be formed of a material, other than a resin, that has lower conductivity than the conductive adhesive layer 12. Further, the first insulating portion 31 and the second insulating portion 41 are not limited to the configuration in which the protruding portion provided on one is fitted into the hole provided on the other, and may be joined to each other by, for example, heat welding, ultrasonic welding, or adhesion using an adhesive, or may have a slide lock mechanism or the like.

Further, one or more of the engaging portion 35, the engaging portion 36, the hole 37, and the hole 38 of the first insulating portion 31 may not be formed. Further, one or more of the claw portion 45, the claw portion 46, the protruding portion 47, and the protruding portion 48 of the second insulating portion 41 may not be provided. The hole 37 of the first insulating portion 31 may not be provided with notches. Further, the protruding portion 47 of the second insulating portion 41 may not be provided with claws at the tip.

Further, the washer 14 is not limited to being provided between the cable terminal 21 and the first insulating portion 31. Further, the washer 14 may be a spring washer or the like. Further, the defibrillator electrode pad 101 is not limited to the configuration including the washer 14, and may have a configuration not including the washer 14. However, since the metal foil 11 and the cable terminal 21 can be brought into close contact with each other by providing the washer 14, it is preferable to provide the washer 14.

As described above, in the defibrillator electrode pad 101 according to the first embodiment of the presently disclosed subject matter, the metal foil 11 is configured to diffuse the electricity transmitted from a defibrillator via the cable 20. Further, the conductive adhesive layer 12 is provided on a part of the first surface 11a of the metal foil 11 and attached to a living body. Further, the base member 15 is provided on the surface of the metal foil 11 opposite to the first surface 11a. Further, the insulating part 16 covers a portion of the first surface 11a of the metal foil 11 where the conductive adhesive layer 12 is not provided, and fixes, to the metal foil 11, the cable terminal 21 provided at the end portion of the cable 20.

As described above, the configuration in which the insulating part 16 covers the exposed portion of the metal foil 11 and fixes the cable terminal 21 to the metal foil 11 makes it possible to avoid the exposure of the metal foil 11 and eliminate the use of a rivet or the like for fixing the cable terminal 21 to the metal foil 11. Thus, the number of components can be reduced.

Further, in the defibrillator electrode pad 101, at least a portion of the insulating part 16 covering the metal foil 11 is made of a resin. With such a configuration, during assembly of the defibrillator electrode pad 101, the insulating part 16 can be manually attached. Thus, installation of large-scale equipment can be avoided, and further, it is possible to significantly reduce production man-hours and ensure safety during assembly.

Further, in the defibrillator electrode pad 101, the insulating part 16 can include the first insulating portion 31 that covers the metal foil 11 and the second insulating portion 41 configured to be fitted to the first insulating portion 31. The metal foil 11 and the cable terminal 21 are sandwiched between the first insulating portion 31 and the second insulating portion 41. With such a configuration, the metal foil 11 and the cable terminal 21 can be more reliably fixed.

Further, in the defibrillator electrode pad 101, the second insulating portion 41 is made of a resin. With such a configuration, the insulating part 16 capable of fixing the metal foil 11 and the cable terminal 21 more reliably can be implemented at low cost. Further, in a case where both the first insulating portion 31 and the second insulating portion 41 are made of a resin, even in a case where moisture from the conductive adhesive layer 12 adheres to at least one of the first insulating portion 31 and the second insulating portion 41, corrosion of the first insulating portion and the second insulating portion can be avoided.

Further, in the defibrillator electrode pad 101, the second insulating portion 41 can include the protruding portion 47 and the protruding portion 48. Further, the first insulating portion 31 can have the hole 37 and the hole 38 into which the protruding portion 47 and the protruding portion 48 of the second insulating portion 41 are inserted, respectively. With such a configuration, the first insulating portion 31 and the second insulating portion 41 can be easily fitted to each other. Further, since the first insulating portion 31 is disposed on a side where the conductive adhesive layer 12 is located and the second insulating portion 41 is disposed on a side opposite to the conductive adhesive layer 12, the protruding portion 47 and the protruding portion 48 of the second insulating portion 41 protrude upward in a state where the conductive adhesive layer 12 faces upward with respect to the base member 15. Therefore, as compared with a case where the protruding portion 47 and the protruding portion 48 for fitting the first insulating portion 31 and the second insulating portion 41 to each other protrude downward, the work of assembling the defibrillator electrode pad 101 can be facilitated.

Further, in the defibrillator electrode pad 101, the second insulating portion 41 can include two or more protruding portions 48. With such a configuration, for example, the insulating part 16 can be prevented from rotating with respect to the metal foil 11 and the base member 15, since two or more protruding portions 48 are disposed to penetrate the base member 15. Further, for example, a position of the cable 20 can be stabilized since the cable 20 is disposed such that the cable 20 passes between the plurality of protruding portions 48.

Further, in the defibrillator electrode pad 101, the first insulating portion 31 and the second insulating portion 41 of the insulating part 16 are subjected to an anti-slip processing on surfaces opposite to the surfaces in contact with the metal foil 11, that is, the outer surfaces 33 and 43.

With such a configuration, for example, in a case where the release sheet attached to the conductive adhesive layer 12 is peeled off, the operator can be prompted to grip the insulating part 16 and the work of peeling off the release sheet can be facilitated. Further, unlike a rivet and a label used in a defibrillator electrode pad in the related art, since the insulating part 16 having a large area capable of covering the exposed portion of the metal foil 11 and having a strength capable of fixing the cable terminal 21 to the metal foil 11 is provided, as described above, a processing for preventing sliding can be performed on the insulating part 16.

Further, in the defibrillator electrode pad 101, the metal foil 11 has a shape having corners, and the first insulating portion 31 and the second insulating portion 41 of the insulating part 16 cover a corner of the metal foil 11. With such a configuration, for example, in a case where the release sheet attached to the conductive adhesive layer 12 is peeled off, it is possible to prompt to peel off the release sheet from a corner portion of the metal foil 11, that is, a portion where it is easy to peel off the release sheet.

Further, in the defibrillator electrode pad 101, in the insulating part 16, an end portion positioned on the center side of the metal foil 11 in a state of covering the metal foil 11, that is, the side 31a of the first insulating portion 31 and the side 41c of the second insulating portion 41 are curved in a direction away from the center of the metal foil 11. With such a configuration, for example, in a case where the operator grips the first insulating portion 31 and the second insulating portion 41 and peels off the release sheet attached to the conductive adhesive layer 12 from the vicinity of the first insulating portion 31, stress concentration on the metal foil 11 can be avoided and damage to the metal foil 11 can be reduced.

Further, the defibrillator electrode pad 101 can further include the washer 14 provided between the first insulating portion 31 and the cable terminal 21. With such a configuration, the metal foil 11 and the cable terminal 21 can be more reliably brought into close contact with each other in a state where the first insulating portion 31 covers the metal foil 11.

### Second Embodiment

### (Overall Configuration)

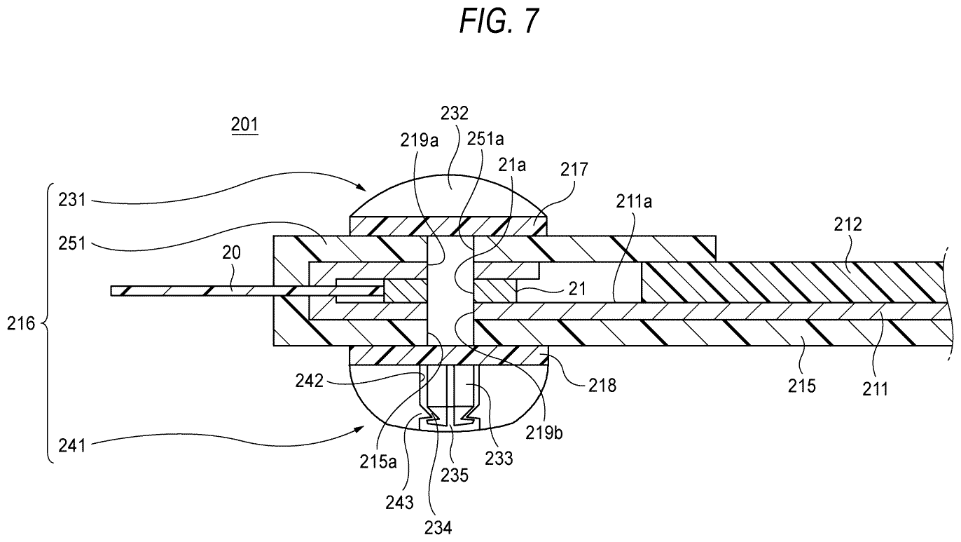

Next, another embodiment of the defibrillator electrode pad of the presently disclosed subject matter will be described. FIG. 7 is a cross-sectional view illustrating a configuration of a defibrillator electrode pad 201 according to a second embodiment of the presently disclosed subject matter. Except for the configuration to be described below, the defibrillator electrode pad 201 has the same configuration as the defibrillator electrode pad 101 according to the first embodiment.

The defibrillator electrode pad 201 can include a metal foil 211, a conductive adhesive layer 212, a base member 215, an insulating part 216, a first washer 217, a second washer 218, and a release sheet, which is not illustrated.

The same as or similarly to the metal foil 11 illustrated in FIG. 1, the metal foil 211 has, for example, a substantially square shape or rectangular shape. The conductive adhesive layer 212 is attached to a part of one surface (hereinafter, referred to as a “first surface 211a”) of the metal foil 211, specifically, a portion excluding one of four corners, and the base member 215 is attached to the other surface of the metal foil 211. Further, the one corner of the metal foil 211 is bent to sandwich the cable terminal 21 provided at the end portion of the cable 20.

The insulating part 216 can include a first snap (first insulating portion) 231, a second snap (second insulating portion) 241, and a covering portion 251. The first snap 231 and the second snap 241 are made of, for example, resin such as POM or PET.

The covering portion 251 is a part of the base member 215 and covers a portion of the first surface 211a of the metal foil 211 to which the conductive adhesive layer 212 is not attached. More specifically, the base member 215 has a substantially square shape or a substantially rectangular shape larger than that of the metal foil 211, and among four corners of the base member 215, a corner corresponding to the bent corner of the metal foil 211 is bent toward the metal foil 211. The bent corner of the base member 215 corresponds to the covering portion 251.

The first snap 231 and the second snap 241 fix the cable terminal 21 to the metal foil 211. More specifically, the first snap 231 can include a head portion 232, and a shaft portion 233 extending from the vicinity of a center of the head portion 232. A notch 235 is formed along an extending direction of the shaft portion 233 at an end portion (hereinafter, referred to as a “tip end”) of the shaft portion 233 opposite to the head portion 232. A fitting groove 234 is formed on an outer periphery of the shaft portion 233.

A hole 242 into which the shaft portion 233 of the first snap 231 is configured to be inserted is formed in the second snap 241. Further, a claw portion 243 configured to be fitted into the fitting groove 234 of the shaft portion 233 of the first snap 231 is provided on an inner wall of the hole 242.

Each of the first washer 217 and the second washer 218 is, for example, a circular member formed of resin. Each of the first washer 217 and the second washer 218 has a circular hole inside. The first washer 217 is provided between the first snap 231 and the covering portion 251. The second washer 218 is provided between the second snap 241 and the base member 215.

Further, a hole 219a is formed in the metal foil 211 on a first snap 231 side at a position facing the hole 21a of the cable terminal 21 in a state where the cable terminal 21 is sandwiched at the corner. Further, in this state, a hole 219b is formed in the metal foil 211 on a second snap 241 side at a position facing the hole 21a of the cable terminal 21.

One hole 251a is formed in the covering portion 251 at a position facing the hole 219a of the metal foil 211. One hole 215a is formed in the base member 215 at a position facing the hole 219b of the metal foil 211.

### (Assembly of Defibrillator Electrode Pad)

During assembly of the defibrillator electrode pad 201, the cable 20 is inserted into the hole formed in the bent portion of the metal foil 211, and into the hole formed in the bent portion of the base member 215. Then, in a state where the cable terminal 21 is covered with a part of the metal foil 211 and the covering portion 251, the shaft portion 233 of the first snap 231 is inserted into the hole of the first washer 217, the hole 251a of the covering portion 251, the hole 219a of the metal foil 211, the hole 21a of the cable terminal 21, the hole 219b of the metal foil 211, and the hole 215a of the base member 215.

Further, the shaft portion 233 of the first snap 231 is inserted into the hole 218a of the second washer 218 and the hole 242 of the second snap 241. At this time, the shaft portion 233 is deformed such that the notch 235 formed at the tip end thereof is narrowed, and in a case where the claw portion 243 of the hole 242 is fitted into the fitting groove 234 of the shaft portion 233, the notch 235 is widened. Accordingly, the first snap 231 and the second snap 241 are fitted to each other, and the assembly of the defibrillator electrode pad 201 is completed.

A plurality of fitting grooves 234 may be formed in the shaft portion 233 of the first snap 231. In this case, the same number of claw portions 243 as the number of fitting grooves 234 are provided on the inner wall of the hole 242 of the second snap 241. In such a configuration, in a case where the first snap 231 and the second snap 241 are fitted to each other, the operator feels a plurality of resistances, and thus it is easy to grasp that the first snap 231 and the second snap 241 are fitted to each other.

A position of the fitting groove 234 formed in the shaft portion 233 of the first snap 231 is not limited to the vicinity of the tip end of the shaft portion 233, and may be formed in the vicinity of a base portion of the notch 235.

The metal foil 211 is not limited to being bent as illustrated in FIG. 7. However, in the case where the metal foil 211 is bent to sandwich the cable terminal 21, the number of electrical contacts between the metal foil 211 and the cable terminal 21 is increased, and contact failure can be prevented. Therefore, the metal foil 211 is preferably bent to sandwich the cable terminal 21.

As described above, in the defibrillator electrode pad 201 according to the second embodiment of the presently disclosed subject matter, the insulating part 216 can include the covering portion 251 that covers the first surface 211a of the metal foil 211. Further, at least the covering portion 251 of the insulating part 216 and the base member 215 are integrally formed. With such a configuration, the number of components can be further reduced, and thus simplification when assembling the defibrillator electrode pad 201 and cost reduction can be realized.

### Modification 1

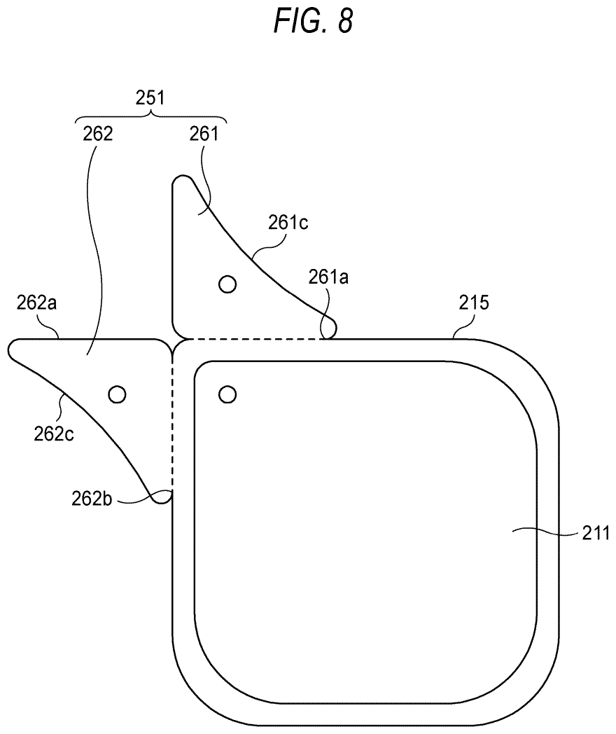

FIG. 8 illustrates Modification 1 of the base member 215 and the covering portion 251 illustrated in FIG. 7. As described above, the base member 215 illustrated in FIG. 7 covers, as the covering portion 251, a part of the metal foil 211 by bending one corner of the substantially square shape or the substantially rectangular shape. Meanwhile, the covering portion 251 according to Modification 1 extends from the base member 215 and is formed integrally with the base member 215.

Specifically, at one corner of the base member 215, a first covering portion 261 and a second covering portion 262 extend as the covering portion 251. The first covering portion 261 has a substantially isosceles right triangular shape. The first covering portion 261 has two sides 261a and 261b sandwiching a right angle corner, and a side 261c facing the right angle corner. The second covering portion 262 has a substantially isosceles right triangular shape. The second covering portion 262 has two sides 262a and 262b sandwiching a right angle corner, and a side 262c facing the right angle corner.

The first covering portion 261 and the second covering portion 262 are provided such that, for example, the side 261a of the first covering portion 261 is in contact with one side of the base member 215, the side 262b of the second covering portion 262 is in contact with the other side of the base member 215, and the right angle corner of the first covering portion 261 and the right angle corner of the second covering portion 262 face each other.

When assembling the defibrillator electrode pad 201, the first covering portion 261 is bent toward a surface of the base member 215 with which the metal foil 211 is in contact in a state where the cable terminal 21 illustrated in FIG. 7 is in contact with the metal foil 211. Further, the second covering portion 262 is bent to overlap the bent first covering portion 261. Accordingly, the metal foil 211 is wrapped by the first covering portion 261 and the second covering portion 262, and exposure of the metal foil 211 can be avoided.

### Modification 2

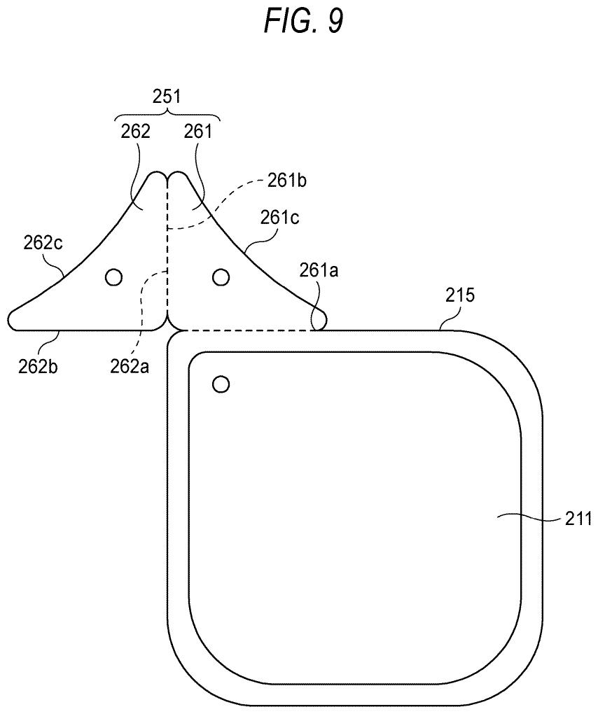

FIG. 9 illustrates Modification 2 of the base member 215 and the covering portion 251 illustrated in FIG. 7. The same as or similarly to the covering portion 251 illustrated in FIG. 8, the covering portion 251 according to Modification 2 can include the first covering portion 261 and the second covering portion 262. The covering portion 251 according to Modification 2 is different from the covering portion 251 illustrated in FIG. 8 in a position of the second covering portion 262. Specifically, the side 262a of the second covering portion 262 is in contact with the side 261b of the first covering portion 261. Further, the right angle corner of the second covering portion 262 and the corner of the base member 215 face each other.

When assembling the defibrillator electrode pad 201, the first covering portion 261 and the second covering portion 262 are bent toward a surface of the base member 215 in contact with the metal foil 211 in a state where the cable terminal 21 illustrated in FIG. 7 is in contact with the metal foil 211. Further, the second covering portion 262 is bent toward a surface of the base member 215 not in contact with the metal foil 211. Accordingly, the base member 215, the metal foil 211, and the cable terminal 21 can be sandwiched between the first covering portion 261 and the second covering portion 262.

As described above, in the defibrillator electrode pad 201 according to Modification 1 and Modification 2 of the second embodiment of the presently disclosed subject matter, the covering portion 251 extends from the base member 215, and is configured to be bent toward the metal foil 211 to cover the metal foil 211. With such a configuration, the defibrillator electrode pad 201 can be assembled more easily.

### Third Embodiment

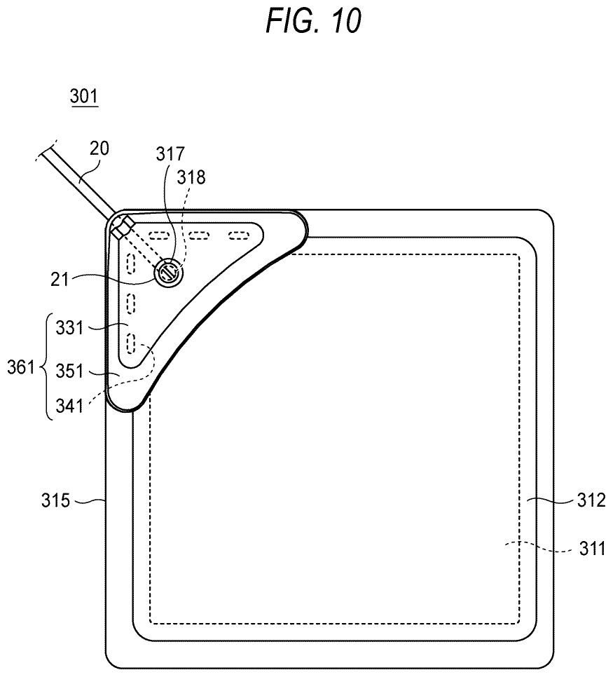

FIG. 10 illustrates a configuration of a defibrillator electrode pad 301 according to a third embodiment of the presently disclosed subject matter. Referring to FIG. 10, the defibrillator electrode pad 301 according to the third embodiment can include a metal foil 311, a conductive adhesive layer 312, a base member 315, an insulating part 316, a first washer 317, a second washer 318, and a release sheet, which is not illustrated.

The metal foil 311, the conductive adhesive layer 312, the base member 315, the first washer 317, and the second washer 318 of the defibrillator electrode pad 301 have the same configurations as the metal foil 211, the conductive adhesive layer 212, the base member 215, the first washer 217, and the second washer 218 of the defibrillator electrode pad 201 illustrated in FIG. 7, respectively.

### (Configuration of Insulating Part)

The insulating part 361 can include a covering portion 351, a first snap (first insulating portion) 331, and a second snap (second insulating portion) 341. For example, the same as or similarly to the covering portion 251 illustrated in FIG. 8, the covering portion 351 extends from the base member 315 to cover the metal foil 311.

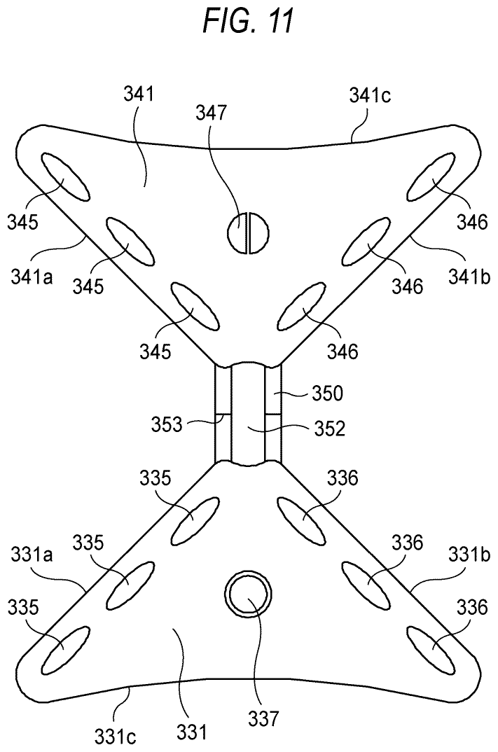

FIG. 11 illustrates a configuration of the first snap 331 and the second snap 341 illustrated in FIG. 10. The first snap 331 and the second snap 341 have larger areas than the first snap 231 and the second snap 241 illustrated in FIG. 7. The first snap 331 and the second snap 341 are provided separately from the base member 315.

More specifically, the same as or similarly to the first insulating portion 31 and the second insulating portion 41 according to the first embodiment illustrated in FIG. 1, each of the first snap 331 and the second snap 341 has, for example, a substantially isosceles right triangular shape, and is made of a resin such as POM or PET. The first snap 331 and the second snap 341 are smaller in area than, for example, the covering portion 351 illustrated in FIG. 10.

The first snap 331 has two sides 331a and 331b sandwiching a right angle corner, and a side 331c facing the right angle corner. Further, the first snap 331 can include a protruding portion 335 formed along the side 331a, a protruding portion 336 formed along the side 331b, and a hole 337 formed near a center of the first snap 331. The first snap 331 illustrated in FIG. 11 can include, for example, three protruding portions 335, three protruding portions 336, and one hole 337.

The second snap 341 has two sides 341a and 341b sandwiching a right angle corner, and a side 341c facing the right angle corner. Further, the second snap 341 can include a protruding portion 345 provided along the side 341a, a protruding portion 346 provided along the side 341b, and a protruding portion 347 that is provided near a center of the second snap 341 and configured to be fitted into the hole 337 of the first snap 331. The second snap 341 illustrated in FIG. 11 can include, for example, three protruding portions 345, three protruding portions 346, and one protruding portion 347.

Further, the first snap 331 and the second snap 341 are integrally formed. Specifically, the first snap 331 and the second snap 341 are connected via a connecting portion 350. A hole 352 through which the cable 20 illustrated in FIG. 10 is inserted is formed in the connecting portion 350. A bent portion 353 is provided near a center of the connecting portion 350. The first snap 331 and the second snap 341 can be overlapped by bending the bent portion 353.

### (Assembly of Defibrillator Electrode Pad)

When assembling the defibrillator electrode pad 301, the second snap 341 is disposed such that the three protruding portions 345 and the three protruding portions 346 of the second snap 341 are located on an outer side of the metal foil 311. Further, in a state where the cable terminal 21 illustrated in FIG. 10 is covered by the metal foil 311 and the covering portion 351, the protruding portion 347 of the second snap 341 is inserted into the hole 21a of the cable terminal 21 and the hole of the second washer 318.

The cable 20 is disposed to pass through the hole 352 of the connecting portion 350. In this state, the bent portion 353 is bent to overlap the first snap 331 and the second snap 341, and the protruding portion 347 of the second snap 341 is fitted into the hole 337 of the first snap 331. Accordingly, the first snap 331 and the second snap 341 are fitted to each other, and the assembly of the defibrillator electrode pad 301 is completed.

As described above, since the first snap 331 and the second snap 341 cover the covering portion 351 with a larger area than the first snap 231 and the second snap 241 illustrated in FIG. 7, the covering portion 351 can be prevented from being separated from the metal foil 311.

Further, the three protruding portions 335 of the first snap 331 and the three protruding portions 345 of the second snap 341 sandwich the base member 315 and the covering portion 351. Further, the three protruding portions 336 of the first snap 331 and the three protruding portions 346 of the second snap 341 sandwich the base member 315 and the covering portion 351. Therefore, the covering portion 351 can be more reliably prevented from being separated from the metal foil 311.

The first snap 331 may not include at least one of the protruding portion 335 and the protruding portion 336. Further, the second snap 341 may not include at least one of the protruding portion 345 and the protruding portion 346.

As described above, in the defibrillator electrode pad 301 according to the third embodiment of the presently disclosed subject matter, the first snap 331 and the second snap 341 are formed integrally. With such a configuration, the number of components can be further reduced.

### Modification

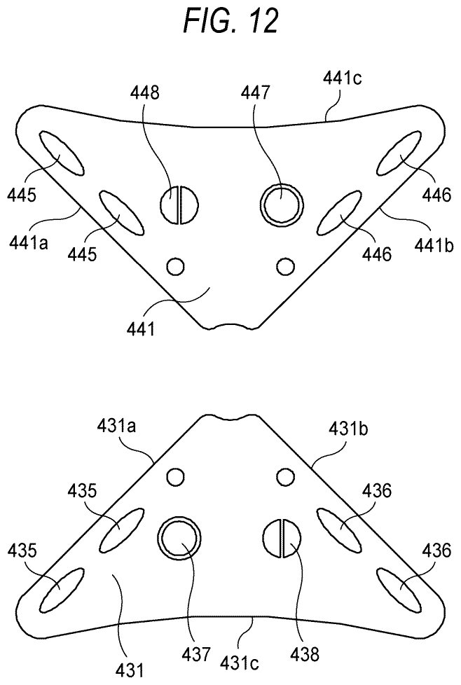

The first snap 331 and the second snap 341 are not limited to the configuration illustrated in FIG. 11, and may not be connected to each other, for example. FIG. 12 illustrates configurations of a first snap (first insulating portion) 431 and a second snap (second insulating portion) 441 according to a modification of the third embodiment of the presently disclosed subject matter. The first snap 431 and the second snap 441 both have a substantially isosceles right triangular shape and have the same shape.

More specifically, the first snap 431 has two sides 431a and 431b sandwiching a right angle corner, and a side 431c facing the right angle corner. Further, the first snap 431 can include a protruding portion 435 formed along the side 431a, a protruding portion 436 formed along the side 431b, a hole 437 formed closer to the side 431a with respect to a center of the first snap 431, and a protruding portion 438 provided closer to the side 431b with respect to the center of the first snap 431. The first snap 431 illustrated in FIG. 12 can include, for example, two protruding portions 435, two protruding portions 436, one hole 437, and one protruding portion 438.

The second snap 441 has two sides 441a and 441b sandwiching a right angle corner, and a side 441c facing the right angle corner. The second snap 441 can include a protruding portion 445 provided along the side 441a, a protruding portion 446 provided along the side 441b, a hole 447 formed closer to the side 441b with respect to a center of the second snap 441, and a protruding portion 448 provided closer to the side 441a with respect to the center of the second snap 441. The second snap 441 illustrated in FIG. 12 can include, for example, two protruding portions 445, two protruding portions 446, one hole 447, and one protruding portion 448.

The protruding portion 438 of the first snap 431 is configured to be fitted into the hole 447 of the second snap 441. The protruding portion 448 of the second snap 441 is configured to be fitted into the hole 437 of the first snap 431.

When assembling the defibrillator electrode pad 301, the first snap 431 and the second snap 441 are overlapped, the protruding portion 438 of the first snap 431 is fitted into the hole 447 of the second snap 441, and the protruding portion 448 of the second snap 441 is fitted into the hole 437 of the first snap 431. Accordingly, the first snap 331 and the second snap 341 can be fitted to each other.

In this way, the first snap 431 and the second snap 441 are used as a common component, and thus the cost can be reduced. Further, since the first snap 431 can include both the hole 437 and the protruding portion 438, and the second snap 441 can include both the hole 447 and the protruding portion 448, the first snap 431 and the second snap 441 can be fitted to each other at two places, and the fitting of the first snap 431 and the second snap 441 can be further strengthened.

Two or more of the first to third embodiments of the presently disclosed subject matter and the modifications of the embodiments may be combined. Although the embodiments of the presently disclosed subject matter have been described above, the technical scope of the presently disclosed subject matter should not be construed as being limited to the description of the present embodiments. The present embodiments are merely an example, and it is understood by those skilled in the art that various modifications of the embodiments are possible within the scope of the disclosed subject matters described in the claims. The technical scope of the present application should be determined based on the scope of the inventions described in the claims and equivalents thereof.

### INDUSTRIAL APPLICABILITY

According to the presently disclosed subject matter, the number of components is reduced and exposure of the metal component is avoided.

1. A defibrillator electrode pad comprising:
a metal foil configured to diffuse electricity transmitted from a defibrillator via a cable;
a conductive adhesive layer configured to be attached to a living body, the conductive adhesive layer being provided on a part of a first surface of the metal foil;
a base member provided on a second surface of the metal foil; and
an insulating part covering a portion of the first surface at which the conductive adhesive layer is not provided, the insulating part fixing, to the metal foil, a cable terminal provided at an end portion of the cable.

2. The defibrillator electrode pad according to claim 1,
wherein at least a portion of the insulating part is made of a resin, the portion of the insulating part covering the metal foil.

3. The defibrillator electrode pad according to claim 1,
wherein the insulating part includes:
a first insulating portion covering the metal foil; and
a second insulating portion configured to be fitted to the first insulating portion, and
the metal foil and the cable terminal are sandwiched between the first insulating portion and the second insulating portion.

4. The defibrillator electrode pad according to claim 3,
wherein the second insulating portion is made of a resin.

5. The defibrillator electrode pad according to claim 3,
wherein the second insulating portion includes one or a plurality of protrusion, and
the first insulating portion has one or a plurality of hole into which the one or plurality of protrusion of the second insulating portion are inserted.

6. The defibrillator electrode pad according to claim 5,
wherein the second insulating portion includes two or more of the protrusions.

7. The defibrillator electrode pad according to claim 3,
wherein the first insulating portion and the second insulating portion are formed integrally.

8. The defibrillator electrode pad according to claim 1,
wherein an anti-slip processing is applied to a surface of the insulating part that is opposite to a surface of the insulating part in contact with the metal foil.

9. The defibrillator electrode pad according to claim 1,
wherein the metal foil has a shape having a corner, and
the insulating part covers the corner of the metal foil.

10. The defibrillator electrode pad according to claim 1,
wherein an end portion of the insulating part is curved in a direction away from a center of the metal foil, the end portion of the insulating part being located on a center side of the metal foil in a state where the insulating part covers the metal foil.

11. The defibrillator electrode pad according to claim 1,
wherein the insulating part includes a covering portion covering the first surface of the metal foil, and
at least the covering portion of the insulating part and the base member are formed integrally.

12. The defibrillator electrode pad according to claim 11,
wherein the covering portion extends from the base member, and
the covering portion is configured to be bent toward the metal foil to cover the metal foil.

13. The defibrillator electrode pad according to claim 1, further comprising:
a washer provided between the insulating part and the cable terminal.
